(19) United States
(12) Patent Application Publication
(10) Pub. No.: US 2024/0057891 A1
(43) Pub. Date: Feb. 22, 2024

(54) ENCODING RESPIRATION FLOW AND VOLUME METRICS USING A MOBILE DEVICE

(71) Applicant: Emercent Technologies LLC, Rochester, MN (US)

(72) Inventor: J. Hunter Downs, III, Rochester, MN (US)

(73) Assignee: Emercent Technologies LLC, Rochester, MN (US)

(21) Appl. No.: 18/385,676

(22) Filed: Oct. 31, 2023

Related U.S. Application Data
(63) Continuation-in-part of application No. 17/186,925, filed on Feb. 26, 2021.
(60) Provisional application No. 63/425,619, filed on Nov. 15, 2022.
(60) Provisional application No. 62/983,267, filed on Feb. 28, 2020.

(51) Int. Cl. A61B 5/09; A61B 5/097; A61B 5/00

(52) U.S. Cl. CPC A61B 5/09; A61B 5/097; A61B 5/7405; A61B 5/7257; A61B 2562/0204

(57) ABSTRACT

Aspects discussed herein relate to spirometers that can be used to measure lung function using a mobile device. A user can breathe into a mouthpiece that forces the user's breath through a spirometric encoding assembly. The spirometric encoding assembly can be coupled to a mobile device using a spirometric encoding adapter. The spirometric encoding assembly can generate one or more outputs based on the user's breath; this data can be measured using one or more sensors of the mobile device. The data captured by the mobile device can be processed to determine the flow rate and/or volume of the user's breath. The flow rate and volume can be used to determine a variety of characteristics of the user's health, lung capacity, and/or for diagnosing medical conditions. A variety of treatment plans can be identified and/or administered based on the diagnosed medical

### CROSS-REFERENCE TO RELATED APPLICATIONS

The instant application claims priority to U.S. Provisional Patent Application No. 63/425,619, titled “Devices for Encoding of Respiration Flow and Volume Metrics using a Mobile Device” and filed Nov. 15, 2022, and is a continuation-in-part of U.S. patent application Ser. No. 17/186,925, titled “Mechanical Device for Auditory Encoding of Respiration Flow and Volume Metrics using a Mobile Device” and filed Feb. 26, 2021, which claims priority to U.S. Provisional Patent Application No. 62/983,267, titled “Mechanical Device for Auditory Encoding of Respiration Flow and Volume Metrics using a Mobile Device” and filed Feb. 28, 2020, the disclosures of which are hereby incorporated by reference in their entirety.

### TECHNOLOGICAL FIELD

Embodiments of the disclosure relate generally to measuring fluid flow and more specifically to spirometry testing.

### BACKGROUND

Spirometry is the measurement of breath, which can be used to determine lung function, such as the amount and/or speed of air that can be inhaled and exhaled by a person.

### SUMMARY

The following presents a simplified summary of various embodiments described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

Embodiments discussed herein relate to spirometers that can be used to measure lung function using a mobile device. Embodiments described herein generally improve the quality, efficiency, and speed of spirometry testing by improving the ability of a system to obtain spirometry data, process the spirometry data, and/or provide treatment for diagnosed medical conditions.

A user can breathe into a mouthpiece that forces the user's breath through a spirometric encoding assembly. The spirometric encoding assembly can be coupled to a mobile device using a spirometric encoding adapter. The mobile device can determine flow rate and/or volume data based on the air flow through the spirometric encoding adapter. The flow rate and volume can be used to determine a variety of characteristics of the user's health, lung capacity, and/or for diagnosing medical conditions. A variety of treatment plans can be identified and/or administered based on the diagnosed medical conditions.

In one embodiment, a spirometric encoding device includes a spirometric encoding assembly including a cylindrically shaped body having an exhaust end and an inlet end, a first encoder assembly, a second encoder assembly, and a turbine, a spirometric encoding adapter including a front panel, at least one side panel, and a rear panel defining an inner cavity having a top end and a lower end, wherein the lower end of the inner cavity includes an inlet port and an outlet port, the spirometric encoding assembly is located between the inlet port and the outlet port of the spirometric encoding adapter, and the top end of the inner cavity is adapted to accept a mobile device, a mouthpiece coupled to the inlet port of the spirometric encoding adapter, and a mobile device coupled to the spirometric encoding adapter and operable to measure a first output from the first encoder assembly, measure a second output from the second encoder assembly, and generate breath data based on the first output and the second output.

In yet another embodiment of the invention, the first encoder assembly includes a magnet embedded in the turbine.

In still another embodiment of the invention, the mobile device is operable to measure the first output using a magnetometer and the mobile device is operable to calculate a rate of air flow based on rotation of the magnet.

In yet still another embodiment of the invention, the second encoder assembly includes a sound encoder assembly including a click assembly that generates a clicking sound at a rate proportional to a rate of air flow.

In yet another additional embodiment of the invention, the second encoder assembly includes a sound encoder assembly including a whistle assembly that generates a signal proportional to a rate of air flow.

In still another additional embodiment of the invention, the mobile device includes a microphone at a first end of the mobile device, the first end of the mobile device is located within the inner cavity, the microphone captures sound generated by the second encoder assembly, and the mobile device is operable to calculate a rate of air flow based on the captured sound.

In yet still another additional embodiment of the invention, the mobile device is operable to generate a reference tone output by a speaker at the first end of the mobile device, the microphone further captures the reference tone output by the speaker, and the mobile device is operable to calculate the rate of air flow based on the captured sound and the reference tone.

In yet another embodiment of the invention, the mouthpiece is coupled to the spirometric encoding adapter via a flexible hose coupled to the inlet port and the mouthpiece.

Yet another embodiment of the invention includes a computer-implemented method including obtaining, using a spirometric encoding device including a spirometric encoding assembly including a magnetic encoder and a sound encoder, air generated by a breathing of a user, capturing, using a magnetometer and based on data captured using the magnetic encoder, a first portion of breath data, capturing, using a microphone and based on data captured using the sound encoder, a second portion of breath data, generating a first signal representation of the first portion of the breath data, generating a second signal representation of the second portion of the breath data, calculating, based on the first signal representation and the second signal representation, a flow rate for the breathing of the user, and calculating, based on the flow rate and a cross sectional area of a breathing tube of the spirometric encoding device, a volume for the breathing of the user.

In yet another embodiment of the invention, the computer-implemented method further includes providing breathing instructions directing the user to breathe through the spirometric encoding device.

In still another embodiment of the invention, the breathing instructions include multiple breathing sessions as part of a single breathing test.

In yet still another embodiment of the invention, the first signal representation is generated based on calculating a Fourier transformation of the first portion of the breath data.

In yet another additional embodiment of the invention, the second signal representation is generated based on calculating a Fourier transformation of the second portion of the breath data.

In still another additional embodiment of the invention, the second signal representation indicates a direction of air flow generated based on the second portion of the breath data and the second portion of the breath data includes a first tone when the air flow is in a first direction and a second tone when the air flow is in a second direction.

Yet another embodiment of the invention includes a non-transitory computer readable medium storing instructions that, when executed by one or more processors, cause the one or more processors to perform steps including obtaining, using a spirometric encoding device including a spirometric encoding assembly including a magnetic encoder and a sound encoder, air generated by a breathing of a user, capturing, using a magnetometer and based on data captured using the magnetic encoder, a first portion of breath data, capturing, using a microphone and based on data captured using the sound encoder, a second portion of breath data, generating a first signal representation of the first portion of the breath data, generating a second signal representation of the second portion of the breath data, calculating, based on the first signal representation and the second signal representation, a flow rate for the breathing of the user, and calculating, based on the flow rate and a cross sectional area of a breathing tube of the spirometric encoding device, a volume for the breathing of the user.

In yet another embodiment of the invention, the instructions further cause the one or more processors to perform steps including providing breathing instructions directing the user to breathe through the spirometric encoding device.

In still another embodiment of the invention, the breathing instructions include multiple breathing sessions as part of a single breathing test.

In yet still another embodiment of the invention, the first signal representation is generated based on calculating a Fourier transformation of the first portion of the breath data.

In yet another additional embodiment of the invention, the second signal representation is generated based on calculating a Fourier transformation of the second portion of the breath data.

In still another additional embodiment of the invention, the second signal representation indicates a direction of air flow generated based on the second portion of the breath data and the second portion of the breath data includes a first tone when the air flow is in a first direction and a second tone when the air flow is in a second direction. These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIG. 1 illustrates an example of an operating environment in which one or more embodiments described herein can be implemented;

FIG. 2 illustrates an example computing device in accordance with one or more embodiments described herein;

FIGS. 3A-C are line drawings of spirometric encoding assemblies in accordance with one or more embodiments described herein;

FIG. 3D is a line drawing of a click assembly in accordance with one or more embodiments described herein;

FIG. 3E is a line drawing of a spirometric encoding assembly in accordance with one or more embodiments described herein;

FIGS. 4A-E are line drawings of spirometric encoding adapters in accordance with one or more embodiments described herein;

FIGS. 5A-C are line drawings of spirometric encoding devices in accordance with one or more embodiments described herein;

FIGS. 6A-C depict flow charts for spirometry testing according to one or more embodiments described herein; and

FIG. 7 depicts a flow chart for diagnosing and treating medical conditions according to one or more embodiments described herein.

### DETAILED DESCRIPTION

In the following description of the various embodiments, reference is made to the accompanying drawings, which form a part hereof, and in which is shown by way of illustration various embodiments in which embodiments of the disclosure can be practiced. It is to be understood that other embodiments can be utilized and structural and functional modifications can be made without departing from the scope of the present disclosure. Embodiments of the disclosure are capable of other embodiments and of being combined, practiced, and/or carried out in various ways. In addition, it is to be understood that the phraseology and terminology used herein are for the purpose of description and should not be regarded as limiting. Rather, the phrases and terms used herein are to be given their broadest interpretation and meaning.

By way of introduction, embodiments discussed herein relate to spirometric encoding adapters and spirometry testing. When a person exhales or inhales into a spirometric encoding adapter, a volume of air is pushed or pulled through a spirometric encoding adapter and either exits the spirometric encoding adapter (on an exhale) or enters the person's mouth (on an inhale). As the air moves through the spirometric encoding adapter, it is forced to rotate in its path using a ring of angled fins on either side of a spirometric encoding assembly located within the spirometric encoding adapter. As the air is rotating, the air strikes a flat turbine or propeller mounted tangentially on an axis fixed between the geometric centers of one or more rotational encoding rings in the spirometric encoding assembly. The turbine and/or propeller can be a two-finned blade, although any number of fins and/or constructions of turbines/propellers can be employed as appropriate. The flow of rotational air causes the turbine blade to rotate at a rate proportional to the flow rate and diameter of the flow pathway.

In many embodiments, the turbine blade, as it rotates, causes a sound to be generated. The sound generated by the turbine blade can be conducted to the microphone of the coupled mobile device via one or more cavities in the spirometric encoding adapter. As the mobile device's microphone can be isolated from most external sounds by the spirometric encoding adapter, the sounds created by the turbine constitute the majority of the auditory signal received by the microphone. This reduces the noise present in the measured data and improves the ability of the mobile device to measure and process the auditory signal. In a number of embodiments, the mobile phone can also generate a reference tone that is mixed with the sounds from the turbine. The mobile phone can capture this audible data and process the audible data to determine flow rate and/or volume.

In a variety of embodiments, one or more of the turbine blades can be magnetically encoded. As the turbine blades rotate, a magnetic field is generated. A magnetometer in a mobile device can be used to detect and measure rotations of the turbine blade as a person blows through the turbine. These rotations are proportional to the force and velocity of the person's exhalation and inhalation. For example, using a 3-axis magnetometer within the mobile device, the complete rotational motion of the turbine blade as well as its direction of rotation (clockwise or counterclockwise) can be captured and used to determine the direction of rotation, indicating whether the person is inhaling or exhaling through the device. The rotation motion can be captured as an angular change from sample to sample with a peak located when the magnetic end of the turbine blade is closest to the magnetometer within the mobile device. Measurement of the peak-to-peak intervals through the breath yield the time course of force and velocity of the person's breathing.

In a variety of embodiments, the rotational speed of the turbine blade can exceed the sampling rate of the magnetometer. For example, magnetometers typically utilized in mobile devices can sample at 240 Hz, which gives a theoretic limit for detecting the rotation speed at 120 rotations per second. This theoretical limit can be exceeded in forced exhalation maneuvers and therefore pose a challenge in the use of this magnetometer approach to spirometry until higher sampling rate magnetometers are developed. However, spirometric encoding adapters in accordance with embodiments are capable of overcoming this limitation in at least one of four ways: (1) magnetic based braking; (2) enlarged turbine blade; (3) gearing; and (4) using multiple sources of data capture.

In the magnetic based braking approach, a second magnet can be placed in the housing of the turbine in proximity to the blade's rotational path. During each rotation, when the two magnets interact, there will be slowing in a predictable and measurable way, thereby creating a braking action. By placing the second magnet on an adjustable or threaded track, the distance between the two magnets, at their closest point of interaction, can be modified to either increase or decrease the braking effect, thereby tuning the speed response of the turbine to air flow range through it to place that airflow range within the accurate magnetometer sampling range.

In the enlarged turbine approach, the radius of the blade from its central hub to its magnet placement can be lengthened to force the blade to sweep out a larger circumference and therefore travel at a lower number of revolutions per second than a shorter blade for the same level of airflow. By projecting the maximal airflow range needed for the application, the length of the blade can be determined to ensure the magnetometer can adequately sample the turbine's rotations.

In the gearing approach, a primary turbine blade can be connected, by reducing gears, to a second rotational arm that contains the magnet that will be sampled by the magnetometer. In a variety of embodiments, the second rotational arm includes the gear itself. By projecting the maximal airflow range needed for the application, the gearing can be determined to ensure the magnetometer can adequately sample the turbine's rotations over that range.

In several embodiments, multiple techniques can be utilized to capture the rotational data from the turbine. For example, when the rotational speed of the turbine is below the sampling threshold of the magnetometer, the magnetometer can be used to measure the rotational speed of the turbine. When the rotational speed of the turbine exceeds the sampling threshold of the magnetometer (e.g. when oversampling occurs), a sound generated during the rotation of the turbine can be measured as described herein.

The mobile phone can capture the rotational data and process the data to determine flow rate and/or volume. The flow rate and/or volume of exhaled and inhaled breath can be used to characterize the person's respiration. The person's respiration can be used for a variety of purposes, including but not limited to training feedback and medical diagnostic purposes. Based on medical diagnostics, one or more treatment plans can be identified and/or administered.

In many embodiments, spirometric encoding devices can be used to administer a number of treatment sessions over time to a particular patient. The patient's historical breathing characteristics can be used to analyze the patient's health over time and/or predict impending medical conditions. For example, if a patient's breathing capability decreases rapidly over a short period of time, it can be predicted that the patient may have contracted a breathing disease (such as COVID-19) or a disease with severe breathing implications such as congestive heart failure. This predictive modeling of a patient's health can be used to recommend and administer treatments while diseases are still in their early stages, thereby limiting the severity of the diseases, improving the efficacy of the treatment of the disease, and/or improving the overall quality of life for the patient.

Operating Environments and Computing Devices

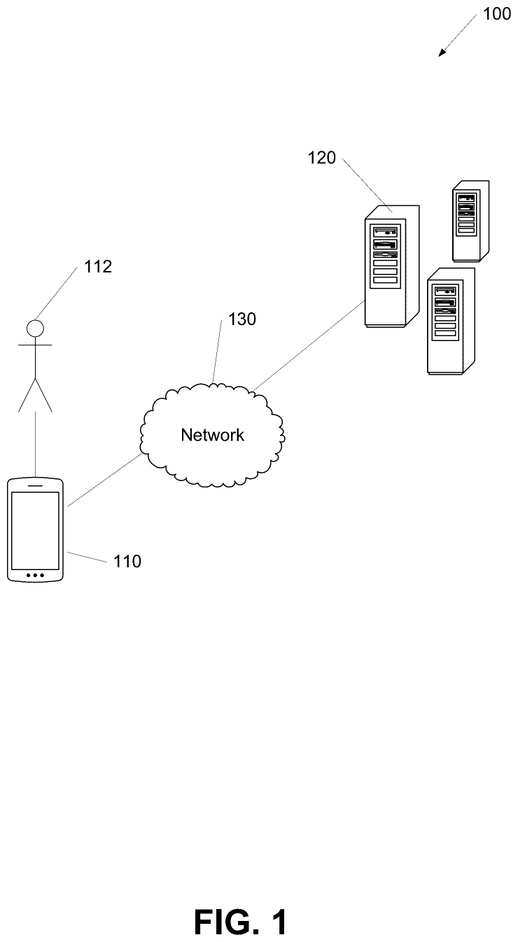

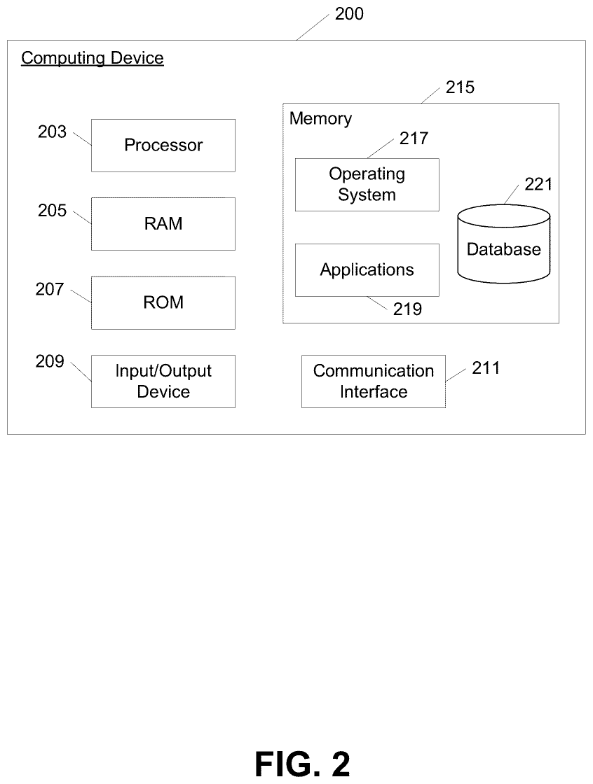

FIG. 1 illustrates an operating environment 100 in accordance with an embodiment of the invention. The operating environment 100 includes at least one mobile device 110 and/or at least one processing server system 120 in communication via a network 130. In a variety of embodiments, the mobile device 110 is coupled to a spirometric encoding adapter 112 worn by a user. It will be appreciated that the network connections shown are illustrative and any means of establishing a communications link between the computers can be used. The existence of any of various network protocols such as TCP/IP, Ethernet, FTP, HTTP and the like, and of various wireless communication technologies such as GSM, CDMA, WiFi, and LTE, 5G, can be used, and the various computing devices described herein can be configured to communicate using any of these network protocols or technologies. Any of the devices and systems described herein can be implemented, in whole or in part, using one or more computing devices described with respect to FIG. 2.

Mobile devices 110 can capture breath data using a spirometric encoding adapter 112 as described herein. Mobile devices 110 can provide data to and/or obtain data from the at least one processing server system 120 as described herein. Processing server systems 120 can store and process a variety of data as described herein. The network 130 can include a local area network (LAN), a wide area network (WAN), a wireless telecommunications network, and/or any other communication network or combinations thereof.

Some or all of the data described herein can be stored using any of a variety of data storage mechanisms, such as databases. These databases can include, but are not limited to relational databases, hierarchical databases, distributed databases, in-memory databases, flat file databases, XML databases, NoSQL databases, graph databases, and/or a combination thereof. The data transferred to and from various computing devices in the operating environment 100 can include secure and sensitive data, such as confidential documents, customer personally identifiable information, and account data. It can be desirable to protect transmissions of such data using secure network protocols and encryption and/or to protect the integrity of the data when stored on the various computing devices. For example, a file-based integration scheme or a service-based integration scheme can be utilized for transmitting data between the various computing devices. Data can be transmitted using various network communication protocols. Secure data transmission protocols and/or encryption can be used in file transfers to protect the integrity of the data, for example, File Transfer Protocol (FTP), Secure File Transfer Protocol (SFTP), and/or Pretty Good Privacy (PGP) encryption. In many embodiments, one or more web services can be implemented within the various computing devices. Web services can be accessed by authorized external devices and users to support input, extraction, and manipulation of data between the various computing devices in the operating environment 100. Web services built to support a personalized display system can be cross-domain and/or cross-platform, and can be built for enterprise use. Data can be transmitted using the Secure Sockets Layer (SSL) or Transport Layer Security (TLS) protocol to provide secure connections between the computing devices. Web services can be implemented using the WS-Security standard, providing for secure SOAP messages using XML encryption. Specialized hardware can be used to provide secure web services. For example, secure network appliances can include built-in features such as hardware-accelerated SSL and HTTPS, WS-Security, and/or firewalls. Such specialized hardware can be installed and configured in the operating environment 100 in front of one or more computing devices such that any external devices can communicate directly with the specialized hardware.

Turning now to FIG. 2, a computing device 200 in accordance with an embodiment of the invention is shown. The computing device 200 can include a processor 203 for controlling overall operation of the computing device 200 and its associated components, including RAM 205, ROM 207, input/output device 209, communication interface 211, and/or memory 215. A data bus can interconnect processor(s) 203, RAM 205, ROM 207, memory 215, I/O device 209, and/or communication interface 211. In some embodiments, computing device 200 can represent, be incorporated in, and/or include various devices such as a desktop computer, a computer server, a mobile device, such as a laptop computer, a tablet computer, a smart phone, any other types of mobile computing devices, and the like, and/or any other type of data processing device.

Input/output (I/O) device 209 can include a microphone, gyroscope, magnetometer, proximity sensor, light sensor, camera, accelerometer, compass, barometer, near field communication (NFC) sensor, fingerprint sensor, pressure sensor, keypad, touch screen, and/or stylus through which the computing device 200 can receive input, and can also include one or more of a speaker for providing audio output and a video display device for providing textual, audiovisual, and/or graphical output. Communication interface 211 can include one or more transceivers, digital signal processors, and/or additional circuitry and software for communicating via any network, wired or wireless, using any protocol as described herein. Software can be stored within memory 215 to provide instructions to processor 203 allowing computing device 200 to perform various actions. For example, memory 215 can store software used by the computing device 200, such as an operating system 217, application programs 219, and/or an associated internal database 221. The various hardware memory units in memory 215 can include volatile and nonvolatile, removable and non-removable media implemented in any method or technology for storage of information such as computer-readable instructions, data structures, program modules, or other data. Memory 215 can include one or more physical persistent memory devices and/or one or more non-persistent memory devices. Memory 215 can include, but is not limited to, random access memory (RAM) 205, read only memory (ROM) 207, electronically erasable programmable read only memory (EEPROM), flash memory or other memory technology, optical disk storage, magnetic cassettes, magnetic tape, magnetic disk storage or other magnetic storage devices, or any other medium that can be used to store the desired information and that can be accessed by processor 203.

Processor 203 can include a single central processing unit (CPU), which can be a single-core or multi-core processor, or can include multiple CPUs. Processor(s) 203 and associated components can allow the computing device 200 to execute a series of computer-readable instructions to perform some or all of the processes described herein. Although not shown in FIG. 2, various elements within memory 215 or other components in computing device 200, can include one or more caches, for example, CPU caches used by the processor 203, page caches used by the operating system 217, disk caches of a hard drive, and/or database caches used to cache content from database 221. For embodiments including a CPU cache, the CPU cache can be used by one or more processors 203 to reduce memory latency and access time. A processor 203 can retrieve data from or write data to the CPU cache rather than reading/writing to memory 215, which can improve the speed of these operations. In some examples, a database cache can be created in which certain data from a database 221 is cached in a separate smaller database in a memory separate from the database, such as in RAM 205 or on a separate computing device. For instance, in a multi-tiered application, a database cache on an application server can reduce data retrieval and data manipulation time by not needing to communicate over a network with a back-end database server. These types of caches and others can be included in various embodiments, and can provide potential advantages in certain implementations of devices, systems, and methods described herein, such as faster response times and less dependence on network conditions when transmitting and receiving data.

Although various components of computing device 200 are described separately, functionality of the various components can be combined and/or performed by a single component and/or multiple computing devices in communication without departing from the invention.

Spirometric Encoding Assemblies and Spirometric Encoding Adapters

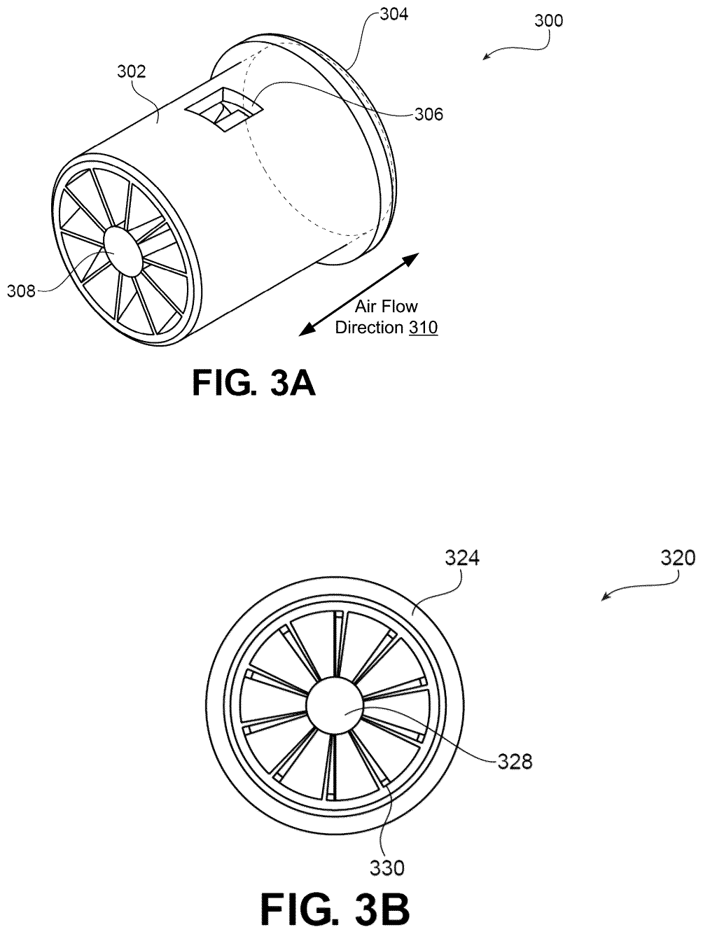

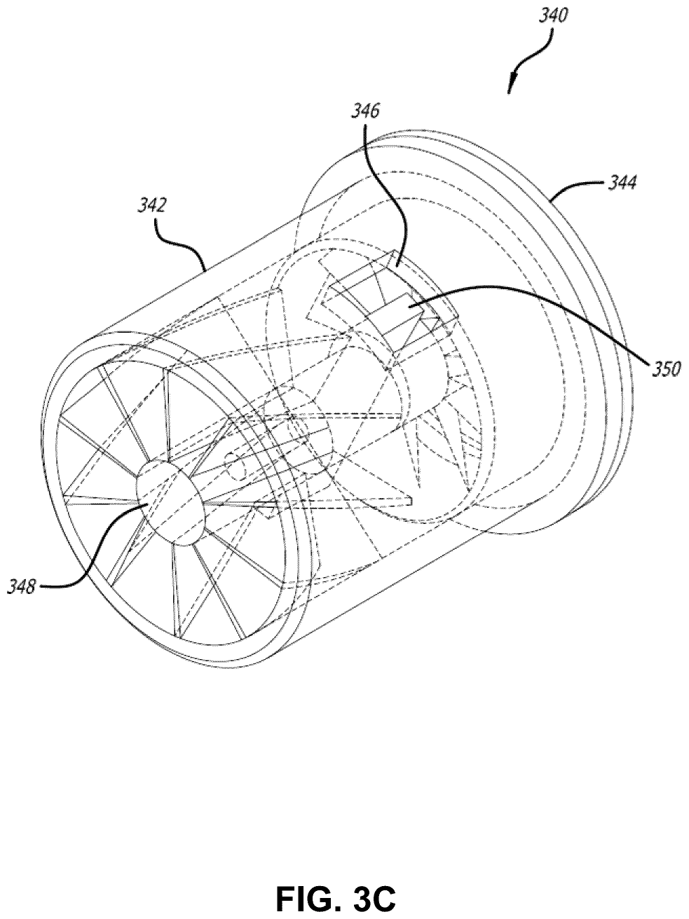

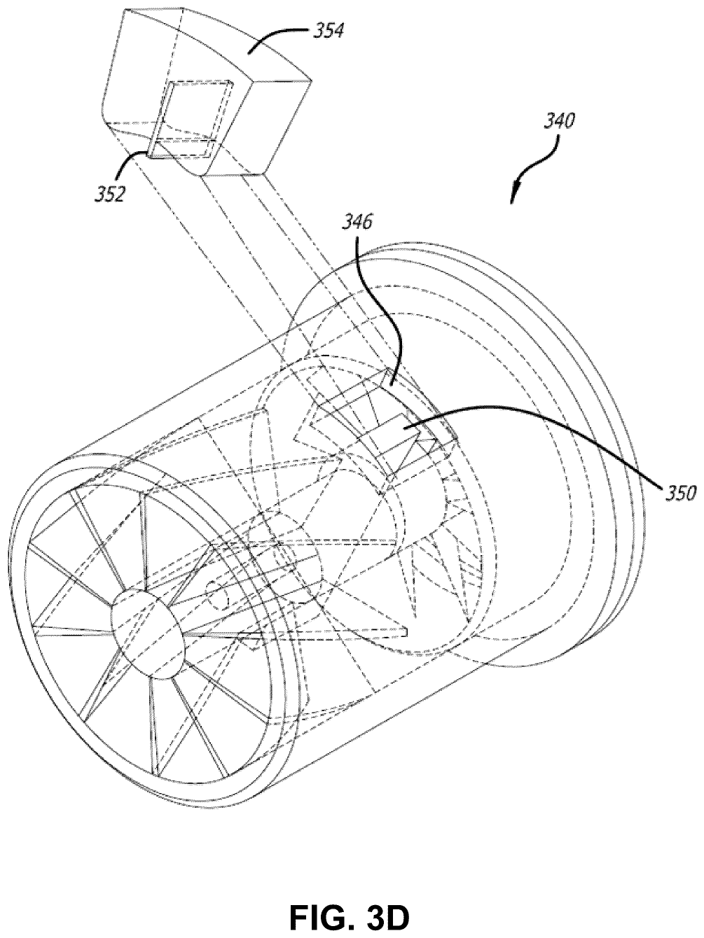

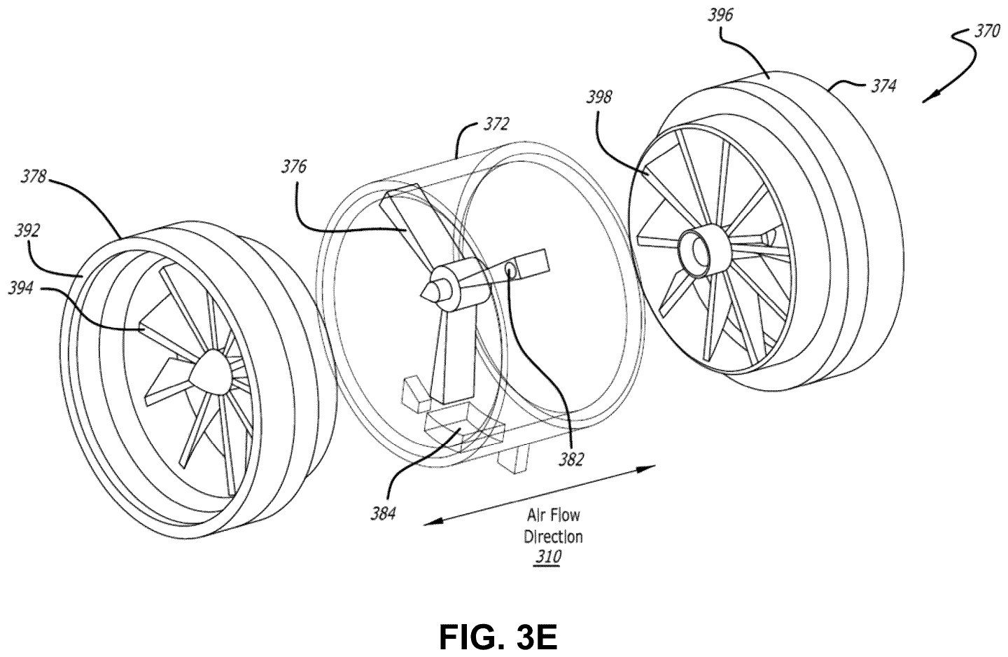

FIGS. 3A-E are line drawings of spirometric encoding assemblies in accordance with one or more embodiments described herein. In FIGS. 3A-E, internal portions of the device are shown in dotted lines.

Turning now to FIG. 3A, a spirometric encoding assembly 300 includes a generally cylindrically shaped body 302, an exhaust end 304, a sound encoder assembly 306, and an inlet end 308. Air can flow through the spirometric encoding assembly 300 as shown by the air flow direction 310.

Turning now to FIG. 3B, an inlet end of a spirometric encoding assembly 320 is shown. The inlet end includes a body 324 and a turbine 328 having one or more fins 330 located within the body 324. Air flowing through the inlet end causes the fins 330 to move, thereby rotating the turbine 328 within the body 324. For example, when a user exhales through the spirometric encoding assembly, the turbine 328 can rotate clockwise as the air flows from the inlet end towards the outlet end. In another example, when the user inhales through the spirometric encoding assembly, the turbine 328 can rotate clockwise as the air flows through the outlet end towards the inlet end. However, it should be noted that the turbine 328 can rotate in any direction in accordance with the requirements of specific applications of the invention. Additionally, the turbine 328 shown in FIG. 3B is designed to rotate along an axis parallel to a longitudinal axis of the body 324. It should be noted that other turbine orientations, such as those turbines that rotate along an axis perpendicular to the longitudinal axis of the body 324, can also be used as appropriate.

Turning now to FIG. 3C, a spirometric encoding assembly 340 is shown. The spirometric encoding assembly 340 includes a body 342, an exhaust end 344, a hole 346, and an inlet end 348. Inside the body 342 is a turbine having a sound encoder assembly 350. The sound encoder assembly 350 can generate a sound as the turbine rotates and the sound generated by the sound encoder assembly 350 can be transmitted through hole 346. The generated sound can include a series of frequency spikes or shifts in acoustic frequency proportional to the flow of air through the spirometric encoding assembly 340. The frequency and phase components of the generated sound are correlated to the flow emanating from the breath flow through path length and turbulence changes of the air as it moves through the spirometric encoding device.

For example, as shown in FIG. 3D, the sound encoder assembly 350 can include a click encoder 354 including a small flexible pin or tab 352 in the path of the turbine blades whereupon the pin or tab 352 makes a distinct and characteristic sound at a particular frequency and interval determined based on the rotational speed of the turbine. The flexible pin or tab 352 can be made out of any of a variety of materials, such as Mylar or any other flexible plastic. In many embodiments, the click encoder 354 can be located within and/or removably inserted into sound encoder assembly 350.

In a variety of embodiments, the sound encoder assembly 350 can utilize a whistle assembly that emits an audible tone at a frequency dependent to flow rates and volume. In a number of embodiments, the spirometric encoding assembly 340 includes a detector for determining the direction of air flow through the turbine. In several embodiments, the detector is a tab mounted asymmetrically within the spirometric encoding assembly 340 such that the tab is less flexible when hit from a first direction as compared to a second direction. In this way, the tab generates a sound at a first frequency when hit from the first direction and a sound at a second frequency when hit from the second direction. The first and second directions can correspond to clockwise and counterclockwise rotation of the turbine.

FIG. 3E is a line drawing of a spirometric encoding assembly in accordance with one or more embodiments described herein. Spirometric encoding assembly 370 includes a generally cylindrically shaped body 372, an exhaust end 374, a magnetic encoder assembly 376, and an inlet end 378. Air can flow through the spirometric encoding assembly 370 as shown by the air flow direction 310. The inlet end 378 can include a body 392 and one or more vanes 394 located within the body 392. The exhaust end 374 can include a body 396 and one or more vanes 398. The vanes 394, 398 can induce a rotational flow to the air flowing through the inlet end 378 and/or outlet end 374. In this way, the air flowing through the body 372 can cause a rotational flow on the magnetic encoder assembly 376. For example, as the air flows through the body 372, the magnetic encoder assembly 376 rotates within the body 372. The magnetic encoder assembly 376 can include one or more magnets 382. A mounting portion 384 formed in the body 372 can be used to mount an encoder (not shown) and/or a braking assembly as described herein. The braking assembly can be used to limit the maximum rotational speed of the magnetic encoder assembly 376 based on the sampling rate of the magnetometer as described herein. In a variety of embodiments, a sound encoder assembly can be installed and/or transmit sound through the mounting portion 384 as described herein.

For example, when a user exhales through the spirometric encoding assembly, the magnetic encoder assembly 376 can rotate clockwise as the air flows from the inlet end towards the outlet end. In another example, when the user inhales through the spirometric encoding assembly, the magnetic encoder assembly 376 can rotate counterclockwise as the air flows through the outlet end towards the inlet end. However, it should be noted that the magnetic encoder assembly 376 can rotate in any direction in accordance with the requirements of specific applications of the invention. Additionally, the magnetic encoder assembly 376 is designed to rotate along an axis parallel to a longitudinal axis of the body 372. It should be noted that other orientations, such as those where magnetic encoder assembly 376 rotates along an axis perpendicular to the longitudinal axis of the body 372, can also be used as appropriate.

Although a variety of spirometric encoding assemblies are shown and described with respect to FIG. 3A-E, any of a variety of other assemblies, including those that include rotating members other than turbines, can be used in accordance with embodiments of the invention.

A spirometric encoding adapter can hold both a mobile device and at least one spirometric encoding assembly. A spirometric encoding adapter can include a single component and/or multiple sub-components. In many embodiments, the spirometric encoding adapter includes a rigid component and a flexible component. A rigid component can house one or more spirometric encoding assemblies and attach to a portion of the flexible component. In a variety of embodiments, the rigid component is hollow to allow sound conduction. One or more holes over the at least one spirometric encoding assembly can allow sounds emanating from the spirometric encoding assembly to travel to the mobile device. The flexible component can couple to the rigid component on a first end and a mobile device on a second end. The flexible component can be hollow to allow sound conduction from the rigid component to the mobile device. In several embodiments, the flexible component conforms to the exterior dimensions of the rigid component and/or mobile device. This can be particularly useful when the rigid component and the mobile device have differing dimensions. The spirometric encoding adapter can be fit with one or more filters to prevent respiratory contaminants, such as dust, dirt, and/or spit, for reaching the mobile device. Resistive load (e.g. breathing difficulty) can be simply added to either the expiratory or inspiratory side of the spirometric encoding adapter using several techniques such as, but not limited to, incorporating rotational flow blocking wheels.

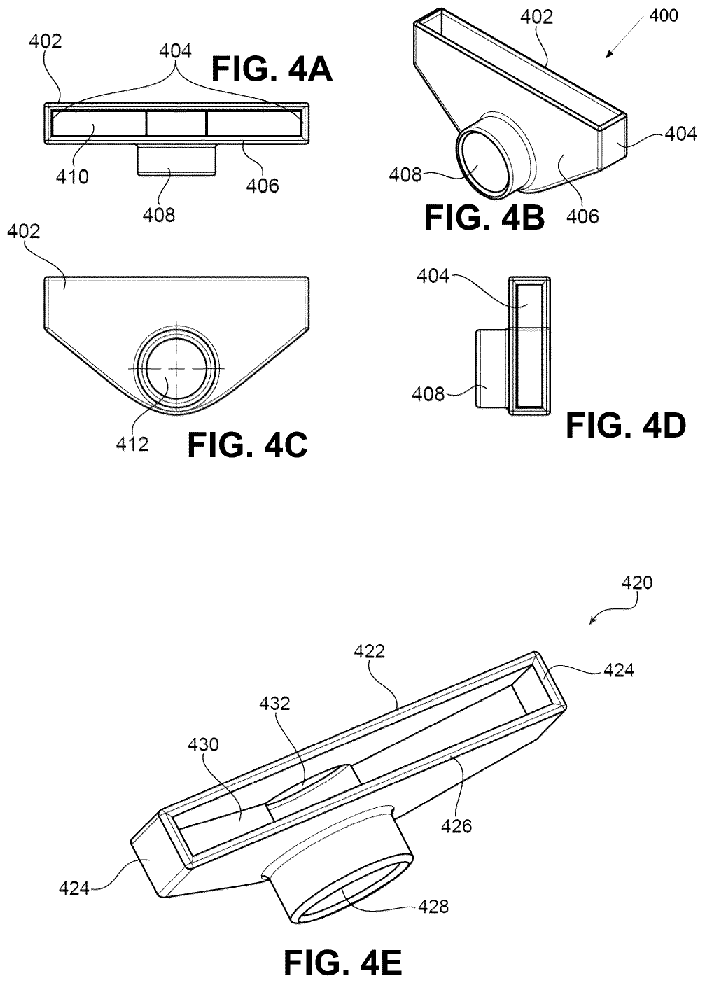

FIGS. 4A-E are line drawings of spirometric encoding adapters in accordance with one or more embodiments described herein. In FIGS. 4A-E, internal portions of the device are shown in dotted lines. Turning now to FIGS. 4A-D, a variety of perspectives of a spirometric encoding adapter 400 are shown. FIG. 4A shows an overhead perspective of the spirometric encoding adapter 400. FIG. 4B shows a three-quarter perspective of the spirometric encoding adapter 400. FIG. 4C shows a rear perspective of the spirometric encoding adapter 400. FIG. 4D shows a lateral perspective of the spirometric encoding adapter 400. The spirometric encoding adapter 400 includes a front panel 402, side panels 404, and a rear panel 406 defining an inner cavity 410 having a top end and a lower end. The top end of the inner cavity 410 defines a square opening, while the lower end of the inner cavity extends to an inlet cavity 412 in the rear panel 406. The inlet cavity 412 is defined by an inlet port 408 in the rear panel 406. In many embodiments, the general shape of the front panel 402, side panels 404, and rear panel 406 are designed to funnel sound generated from a spirometric encoding assembly located in the inlet cavity 412 up through the inner cavity 410 toward the top end of the inner cavity 410.

Turning now to FIG. 4E, a second perspective of a spirometric encoding adapter 420 is shown. The spirometric encoding adapter 420 includes a front panel 422, side panels 424, and a rear panel 426 defining an inner cavity 430 having a top end and a lower end. The rear panel 426 includes an inlet port 428 that allows access to the inner cavity 430 at the lower end of the inner cavity 430. The front panel 422 includes an outlet port 432 that allows access to the inner cavity 430 at the lower end of the inner cavity. The inlet port 428 and outlet port 432 are generally aligned with each other such that air can flow through the inlet port 428 and out the outlet port 432 and vice versa. In a variety of embodiments, a spirometric encoding assembly can be inserted into the outlet port 432 and/or inlet port 428 and held in place within the spirometric encoding adapter 420. A mouthpiece (not shown) or other hose can be coupled to the inlet port 428 to allow for air (or any other fluid) to move through the spirometric encoding assembly as described herein.

In several embodiments, a variety of devices can be coupled to the inlet port and/or the outlet port of the spirometric encoding adapter. For example, a breathing gas source can be coupled to the outlet port, the breathing gas source storing a breathing gas mix of a known mixture of gases (e.g. 70% nitrogen and 30% oxygen, 100% oxygen, or any other gas mixture or combination of gas mixtures as appropriate). As the patient inhales and exhales through the spirometric encoding adapter, the patient will breathe in the breathing gas mix. After completing the breathing tests, the amount of breathing gas mix remaining in the breathing gas source can be measured and the overall breathing capability of the user can be measured. In a variety of embodiments, the breathing capability of the user can be calculated based on the amount of oxygen used by the patient during the breathing exercises.

A mouthpiece and a mobile device can be connected to the spirometric encoding adapter to form a spirometric encoding device. The spirometric encoding device can include an expiratory entrance/inspiratory exit tube, expiratory breath rotational encoding fins, a turbine blade on an axis, inspiratory breath rotational encoding fins, a spirometric encoding assembly, and an expiratory exit/inspiratory entrance tube. The mouthpiece can be coupled to a (flexible) tube having a particular diameter. The tube can be coupled to the spirometric encoding adapter. In this way, the tube couples the mouthpiece to the spirometric encoding adapter. The mouthpiece and/or tube can be constructed out of any material, such as a clear plastic, and can be of any size as appropriate. A user can inhale or exhale air through the mouthpiece, which draws air in from or exhales air through the spirometric encoding adapter.

In a variety of embodiments, this rotation causes a spirometric encoding assembly located in the spirometric encoding adapter to generate a magnetic field that can be measured using a magnetometer located in the spirometric encoding adapter. In many embodiments, this magnetic field data can be processed to determine flow rates and/or a variety of other data as described in more detail herein.

In several embodiments, this rotation causes a spirometric encoding assembly located in the spirometric encoding adapter to emit a sound that can be recorded by a microphone located in the spirometric encoding adapter. This sound can be processed to determine flow rates and/or a variety of other data as described in more detail herein.

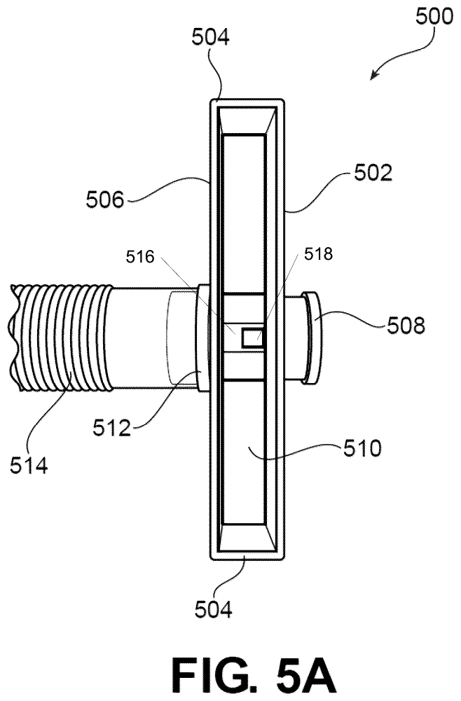

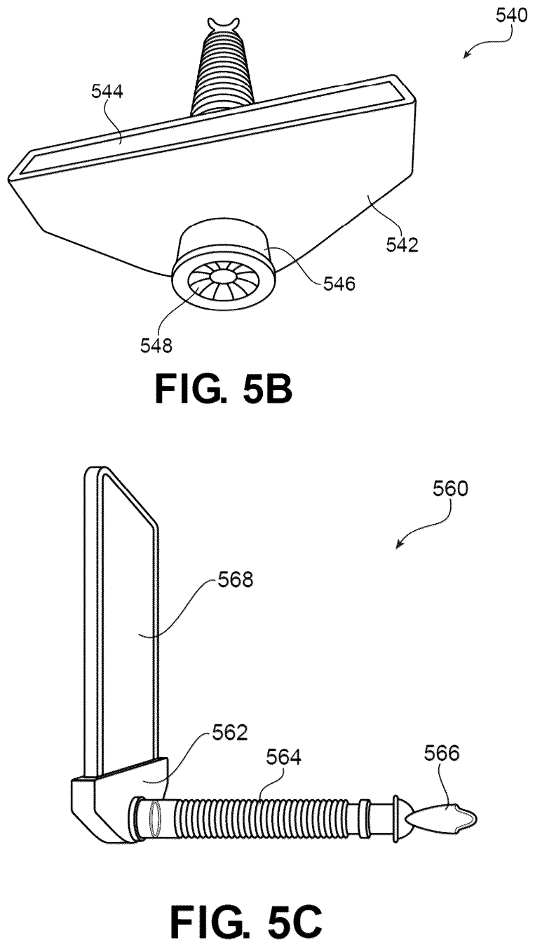

FIGS. 5A-C are line drawings of spirometric encoding devices in accordance with one or more embodiments described herein. In FIGS. 5A-C, internal portions of the device are shown in dotted lines. Turning now to FIG. 5A, a spirometric encoding adapter 500 is shown. The spirometric encoding adapter 500 includes a front panel 502, side panels 504, and a rear panel 506 defining an inner cavity 510 having a top end and a lower end. The top end of the inner cavity 510 defines a generally square or rectangular opening, while the lower end of the inner cavity extends to an inlet port 512 in the rear panel 506 and an outlet port 508 in front panel 502. The inlet port 512 is coupled to a hose or mouthpiece 514. Located in the lower end of the spirometric encoding adapter is a spirometric encoding assembly 516 having a hole 518 oriented toward the top end of the inner cavity 510. It should be noted that the spirometric encoding assembly can be installed in the spirometric encoding adapter in any orientation in accordance with the requirements of particular applications of the invention. The spirometric encoding assembly 516 can be held in place by the inlet port 512 and/or outlet port 508, such as by a friction fit or any applicable fastener.

Turning now to FIG. 5B, a front perspective view of a spirometric encoding adapter 540 is shown. The spirometric encoding adapter 540 has a front wall 542 with an outlet port 546 at the lower end of the spirometric encoding adapter 540. Outlet port 546 has a spirometric encoding assembly 548 installed. Also shown is the top end of the inner cavity 544 defined by the panels of spirometric encoding adapter 540 as described herein.

Turning now to FIG. 5C, a side view of a spirometric encoding device is shown. The spirometric encoding device 560 includes a spirometric encoding adapter 562 coupled to a flexible hose 564, which is coupled to a mouthpiece 566. A mobile device 568 is coupled to the spirometric encoding adapter 562.

Although a variety of spirometric encoding adapters are shown and described with respect to FIGS. 4A-E and 5A—C, any of a variety of other adapters, including those that fit directly into a user's mouth without the use of a hose, can be used in accordance with embodiments of the invention.

Measuring Respiration and Administering Treatments

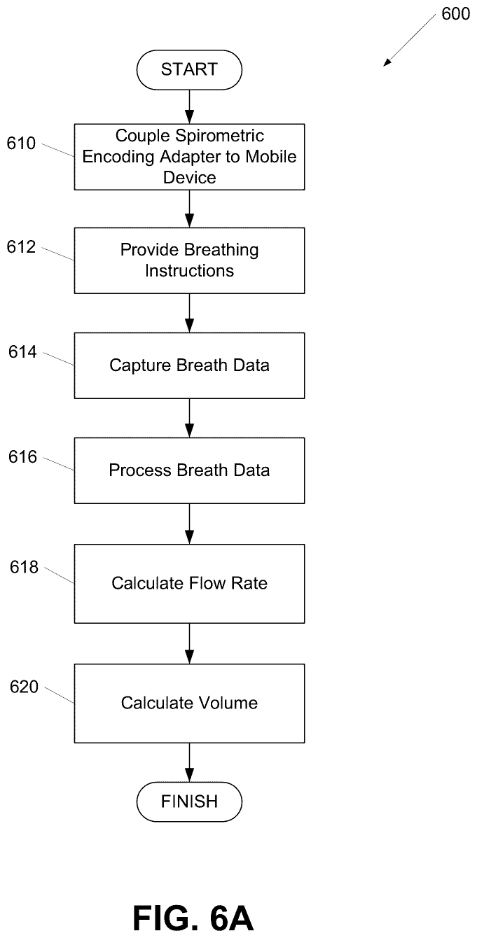

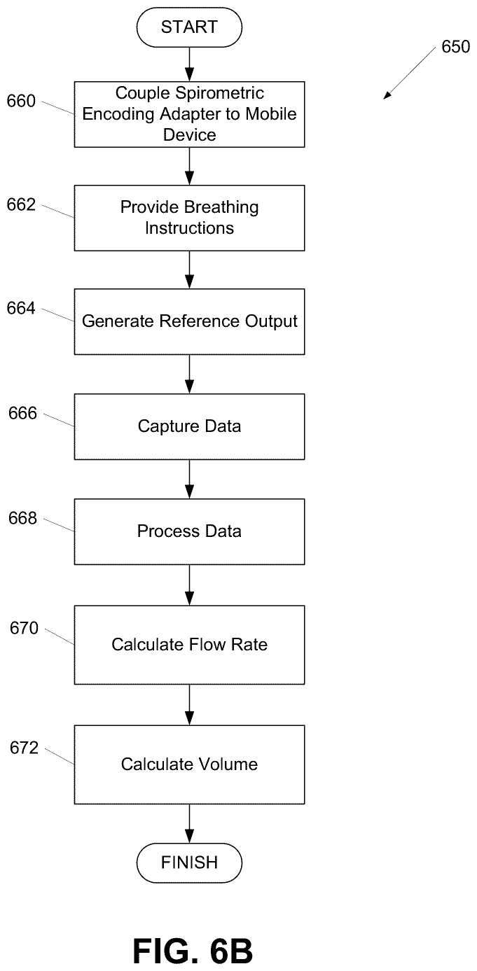

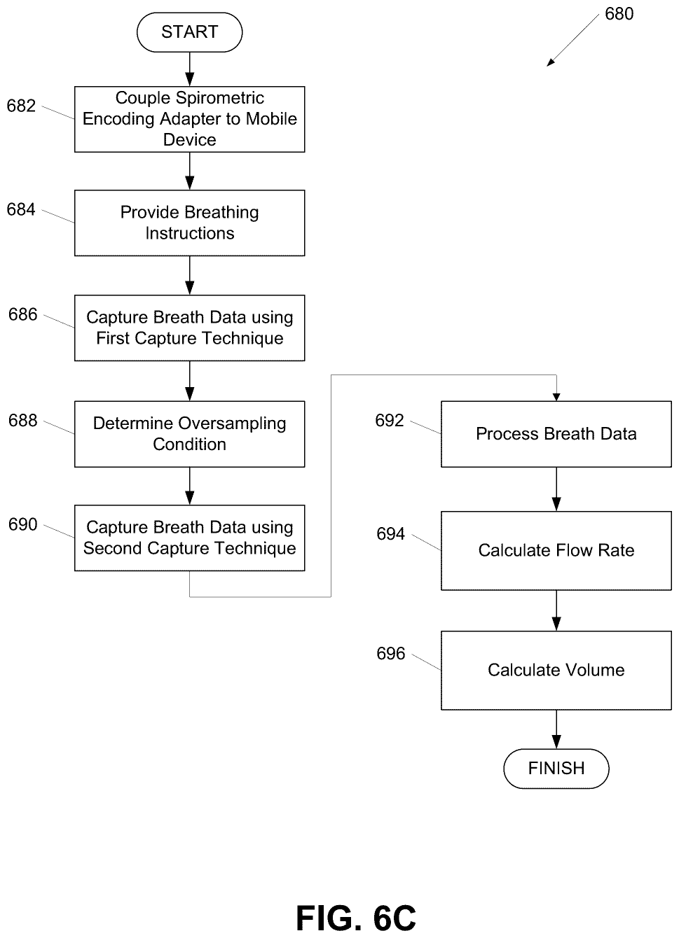

FIG. 6A depicts a flow chart for spirometry testing according to one or more embodiments described herein. Some or all of the steps of process 600 can be performed using any of the computing devices and/or combination thereof described herein. In a variety of embodiments, some or all of the steps described below can be combined and/or divided into sub-steps as appropriate.

A spirometric encoding adapter can be coupled (610) to a mobile device. Breathing instructions can be provided (612). The breathing instructions can be provided as visual and/or audible notifications from the mobile device. The breathing instructions can indicate when and/or how hard a person should breathe into the mouthpiece. The breathing instructions can indicate multiple sessions of breathing to be performed as part of a single test.

Breath data can be captured (614). The breath data can be generated by a spirometric encoding assembly. The breath data can be captured using a one or more sensors and/or input/output devices of the mobile device. In a number of embodiments, the breath data is captured using a magnetometer measuring a magnetic field induced by a rotating magnet in the spirometric encoding assembly. In a variety of embodiments, the breath data is captured using a microphone measuring an audible tone created by the spirometric encoding assembly.

Breath data can be processed (616). The breath data can be processed using any of a variety of techniques, such as a Fourier frequency analysis. In a variety of embodiments, the processing of the breath data includes determining the direction of the air flow. For example, the direction of air flow can be encoded by using flow detector mounted within the spirometric encoding assembly such that the detector generates a different output (e.g. magnetic field, sound, frequency, and/or the like) depending on rotational direction of the turbine. The differences in the output can be used to identify the rotational direction of the turbine, which is directly related to the direction in which the air moving through the turbine is moving.

A flow rate can be calculated (618) and a volume can be calculated (620). The processed breath data can be used to calculate the rate of the turbine's rotation, which can be proportional to the air flow rate. This information, combined with the cross sectional area of the flow tube, can compute air flow and volume of the person's breath. In a number of embodiments, the difference between a reference output, such as a reference tone for a sound encoded output and/or a reference magnetic field for a magnetic encoded output, for the overall spirometric encoding device and the generated output is used to calculate the encoded breath flow of the patient. In several embodiments, the rate at which air flow and/or volume can be calculated is determined based on a sampling rate of the sensor (such as, but not limited to, 240 Hz), a sample window size used for the frequency analysis (such as, but not limited to 2048 samples), the number of blades in the turbine (such as 2), and/or the rate of the turbine's rotation (such as 20 Hz).

FIG. 6B depicts a flow chart for spirometry testing using a reference signal according to one or more embodiments described herein. Some or all of the steps of process 650 can be performed using any of the computing devices and/or combination thereof described herein. In a variety of embodiments, some or all of the steps described below can be combined and/or divided into sub-steps as appropriate.

A spirometric encoding adapter can be coupled (660) to a mobile device and breathing instructions can be provided (662) as described herein. A reference output can be generated (664). The reference output can be generated by the mobile device and/or output by the mobile device. For example, the reference output can be an audible reference tone output by a speaker of the mobile device. In another example, the reference output can include a reference magnetic field. In a variety of embodiments, the reference tone is a square wave tone. However, it should be noted that any output can be used as appropriate to the requirements of specific applications of the invention. In many embodiments, the reference output is generated at a known (e.g. fixed) frequency. In several embodiments, the reference output is generated at a frequency dependent on the speed at which the user is breathing and/or output generated by the spirometric encoding assembly.

Data can be captured (666) as described herein. Data can be processed (668). The data can be processed using a variety of techniques appropriate to the type of data captured as described herein. In a variety of embodiments, the data can be processed by heterodyning the reference output and the output generated by the spirometric encoding assembly. In many embodiments, the data can be heterodyned by mixing the reference output and the output generated by the spirometric encoding assembly to generate one or more beat signals. The frequency of the beat signals can be equal to the difference between the two frequencies and/or equal to the sum of the two frequencies. In a variety of embodiments, a variety of other frequencies that are multiples of the beat frequencies (e.g. harmonic frequencies) are generated. The beat frequencies and/or the harmonic frequencies can be filtered using any of a variety of filters as appropriate to the requirements of specific applications of the invention. In a variety of embodiments, the processing of the output generated by the spirometric encoding assembly includes determining the direction of the air flow. For example, the direction of air flow can be encoded by using flow detector mounted within the spirometric encoding assembly such that the detector generates a different output, such as different sounds and/or a different magnetic field, depending on rotational direction of the turbine. The difference in output (e.g. difference in the frequency generated by the detector) can be used to identify the rotational direction of the turbine, which is directly related to the direction in which the air moving through the turbine is moving.

A flow rate can be calculated (670) and a volume can be calculated (672). The frequency of the beat signal can be used to calculate the rate of the turbine's rotation, which can be proportional to the air flow rate. This information, combined with the cross sectional area of the flow tube, can compute air flow and volume of the person's breath. In a number of embodiments, the difference between a reference output, such as a reference tone for a sound encoded output and/or a reference magnetic field for a magnetic encoded output, for the overall spirometric encoding device and the generated output is used to calculate the encoded breath flow of the patient. In several embodiments, the rate at which air flow and/or volume can be calculated is determined based on a sampling rate of the sensor (such as, but not limited to, 240 Hz), a sample window size used for the frequency analysis (such as, but not limited to 2048 samples), the number of blades in the turbine (such as 2), and/or the rate of the turbine's rotation (such as 20 Hz).

In several embodiments, multiple types of outputs can be sampled in order to generate breath data, thereby allowing for a more accurate measurement of the breath of the patient. For example, measuring the rotation of the turbine using a magnet can be more accurate at low flow rates than measuring sound data. However, the magnetometer in most mobile devices has a relatively low sampling rate relative to the speed of rotation of the turbine used in a variety of embodiments of the invention. This results in a sharp loss in accuracy due to sampling problems at higher flow rates. This problem can be reduced and/or eliminated by switching to a second sampling technique that does not suffer from the oversampling condition. For example, at higher rotational speeds, a sound output of the spirometric encoder can be accurately measured using a microphone of the mobile device. In this way, accurate breath data can be generated across a wider range of flow rates than a single technique.

FIG. 6C depicts a flow chart for spirometry testing using multiple capture techniques according to one or more embodiments described herein. Some or all of the steps of process 680 can be performed using any of the computing devices and/or combination thereof described herein. In a variety of embodiments, some or all of the steps described below can be combined and/or divided into sub-steps as appropriate.

A spirometric encoding adapter can be coupled (682) to a mobile device and breathing instructions can be provided (684) as described herein. Breath data can be captured (686) using a first capture technique. For example, the first capture technique can include measuring a magnetic field generated by the rotation of a turbine as described herein. An oversampling condition can be determined (688). The oversampling condition can be related to the first capture technique. In a variety of embodiments, the oversampling condition can be caused by the rotation speed of the turbine exceeding the sample rate of the sensor measuring the output of the spirometric encoding assembly. For example, the oversampling condition can be caused by a magnet embedded in the turbine of the spirometric encoding assembly exceeding half the sample rate of a magnetometer in the mobile device. Breath data can be captured (690) using a second capture technique. Switching from the first sampling technique to the second technique can be based on the determination of the oversampling condition. For example, when the rotational speed of the turbine exceeds the sampling rate of the magnetometer, the output of the spirometric encoding assembly can be measured based on a sound generated during the rotation of the turbine as described herein.

Breath data can be processed (692) as described herein. In many embodiments, the processed breath data includes a portion of data captured using the first capture technique and a portion of data captured using the second capture technique. A flow rate can be calculated (694) and a volume can be calculated (696) as described herein. In several embodiments, the flow rate and/or volume can be calculated using techniques applicable to the capture technique used to generate a particular portion of the breath data. For example, if a first portion of the breath data is captured using a magnetometer and a second portion of the breath data is captured using a microphone, a first processing technique specific to breath data captured using a magnetometer can be used to process the first portion of the breath data while a second processing technique specific to breath data captured using a microphone can be used to process the second portion of the breath data.

Although a variety of techniques for calculating flow rates and volumes are shown and described with respect to FIG. 6A-C, any of a variety of other techniques, including those that utilize alternative techniques for generating a output using a spirometric encoding adapter and those that use more than two capture techniques for capturing breath data, can be used in accordance with embodiments of the invention.

For example, the above has been described with respect to spirometric encoding adapters that generate magnetic and/or audible data. However, the spirometric encoding adapter can generate a variety of data that represents the air flow through the spirometric encoding adapter. In a number of embodiments, the spirometric encoding assembly generates visual data, such as visible light, infrared, lasers, and/or the like, that can be captured using a camera of the mobile device. For example, visible light can be generated using a flash module of the mobile device. The visible light can be modified by the rotation of the turbine within the spirometric encoding assembly and can be captured using the camera of the mobile device. In many embodiments, the flash and the camera on the mobile device are coupled to the turbine by fiber optic cables such that the flash, in continuous mode (e.g. a flashlight mode) is transmitted through the fiber optic cable to illuminate the rotating turbine blade. In a number of embodiments, the turbine blade is translucent with varying degrees of opacity. The light passing through the blade can be transmitted via a second fiber optic cable back to the camera. The camera can capture the light emitted from the second fiber optic cable at a particular sampling rate. For example, the camera can be configured to capture at its highest frame rate, which can be over 240 fps. However, it should be noted that any capture rate can be utilized as appropriate to the requirements of specific applications of embodiments of the invention. The varying opacities in the turbine blade (which may have an outer wheel to enhance the varying opacities effect) can be used to code the direction and speed of the rotation of the blades. As described above, the speed and direction of the rotation of the blades is proportional to the airflow velocity and direction. In many embodiments, various braking assemblies can be used to limit the maximum rotational speed of the blades based on the capture rate of the camera as described herein. In many embodiments, this visual data can be used to generate breath data, and the processing of the breath data can include processing some or all of the breath data using techniques specific to breath data captured using a camera.

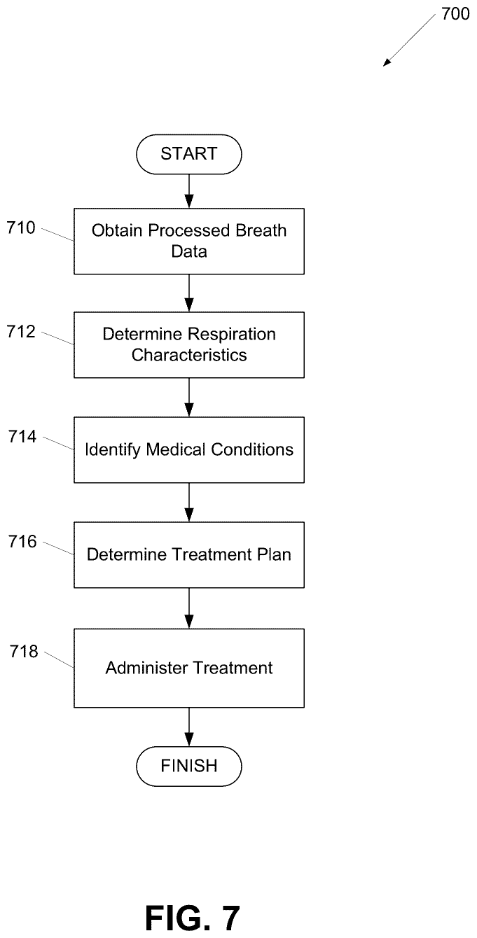

FIG. 7 depicts a flow chart for diagnosing and treating medical conditions according to one or more embodiments of the disclosure. Some or all of the steps of process 700 can be performed using any of the computing devices and/or combination thereof described herein. In a variety of embodiments, some or all of the steps described below can be combined and/or divided into sub-steps as appropriate.

Processed breath data can be obtained (710). The processed breath data can include air flow and/or volume for a person as described herein. The processed breath data can include results from multiple sessions of a person breathing, either concurrently in time or over a period of several days, weeks, months, and/or years. Respiration characteristics can be determined (712), medical conditions can be identified (714), and treatment plans can be determined (716). For example, breathing patterns can be used to determine conditions such as asthma, pulmonary fibrosis, cystic fibrosis, and chronic obstructive pulmonary disease (COPD). In several embodiments, treatments can be administered (718). For example, the effectiveness of a person's asthma medication can be measured over a period of time and, if the efficacy of the medication is below a threshold value, a different asthma medication and/or different dose of the current asthma medication can be recommended and administered to the person in a therapeutically effective dose. In a second example, a person can be diagnosed with COPD and one or more medications (such as bronchodilators and/or corticosteroids) can be prescribed and/or administered to the person in a therapeutically effective dose. The therapeutically effective dose can be determined based on the processed breath data for the person such that the recommended and/or administered treatment is personalized to the person.

Although a variety of techniques for diagnosing and treating medical conditions are shown and described with respect to FIG. 7, any of a variety of other techniques, including those that provide treatment plans including breathing and/or other non-pharmaceutical treatments, can be used in accordance with embodiments of the invention.

One or more embodiments discussed herein can be embodied in computer-usable or readable data and/or computer-executable instructions, such as in one or more program modules, executed by one or more computers or other devices as described herein. Generally, program modules include routines, programs, objects, components, data structures, and the like that perform particular tasks or implement particular abstract data types when executed by a processor in a computer or other device. The modules can be written in a source code programming language that is subsequently compiled for execution, or can be written in a scripting language such as (but not limited to) HTML or XML. The computer executable instructions can be stored on a computer readable medium such as a hard disk, optical disk, removable storage media, solid-state memory, RAM, and the like. As will be appreciated by one of skill in the art, the functionality of the program modules can be combined or distributed as desired in various embodiments. In addition, the functionality can be embodied in whole or in part in firmware or hardware equivalents such as integrated circuits, field programmable gate arrays (FPGA), and the like. Particular data structures can be used to more effectively implement one or more embodiments discussed herein, and such data structures are contemplated within the scope of computer executable instructions and computer-usable data described herein. Various embodiments discussed herein can be embodied as a method, a computing device, a system, and/or a computer program product.

Although the present invention has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive. It will be evident to the annotator skilled in the art to freely combine several or all of the embodiments discussed here as deemed suitable for a specific application of the invention. Throughout this disclosure, terms like “advantageous”, “exemplary” or “preferred” indicate elements or dimensions which are particularly suitable (but not essential) to the invention or an embodiment thereof, and may be modified wherever deemed suitable by the skilled annotator, except where expressly required. Accordingly, the scope of the invention should be determined not by the embodiments illustrated, but by the appended claims and their equivalents.

What is claimed is:

1. A spirometric encoding device, comprising:
a spirometric encoding assembly comprising a cylindrically shaped body having an exhaust end and an inlet end, a first encoder assembly, a second encoder assembly, and a turbine;
a spirometric encoding adapter comprising a front panel, at least one side panel, and a rear panel defining an inner cavity having a top end and a lower end, wherein the lower end of the inner cavity comprises an inlet port and an outlet port, the spirometric encoding assembly is located between the inlet port and the outlet port of the spirometric encoding adapter, and the top end of the inner cavity is adapted to accept a mobile device;
a mouthpiece coupled to the inlet port of the spirometric encoding adapter; and
a mobile device coupled to the spirometric encoding adapter and operable to:
measure a first output from the first encoder assembly;
measure a second output from the second encoder assembly; and
generate breath data based on the first output and the second output.

2. The spirometric encoding device of claim 1, wherein the first encoder assembly comprises a magnet embedded in the turbine.

3. The spirometric encoding device of claim 2, wherein the mobile device is operable to measure the first output using a magnetometer and the mobile device is operable to calculate a rate of air flow based on rotation of the magnet.

4. The spirometric encoding device of claim 1, wherein the second encoder assembly comprises a sound encoder assembly comprising a click assembly that generates a clicking sound at a rate proportional to a rate of air flow.

5. The spirometric encoding device of claim 1, wherein the second encoder assembly comprises a sound encoder assembly comprising a whistle assembly that generates a signal proportional to a rate of air flow.

6. The spirometric encoding device of claim 1, wherein the mobile device comprises a microphone at a first end of the mobile device, the first end of the mobile device is located within the inner cavity, the microphone captures sound generated by the second encoder assembly, and the mobile device is operable to calculate a rate of air flow based on the captured sound.

7. The spirometric encoding device of claim 6, wherein:
the mobile device is operable to generate a reference tone output by a speaker at the first end of the mobile device;
the microphone further captures the reference tone output by the speaker; and
the mobile device is operable to calculate the rate of air flow based on the captured sound and the reference tone.

8. The spirometric encoding device of claim 1, wherein the mouthpiece is coupled to the spirometric encoding adapter via a flexible hose coupled to the inlet port and the mouthpiece.

9. A computer-implemented method, comprising:
obtaining, using a spirometric encoding device comprising a spirometric encoding assembly comprising a magnetic encoder and a sound encoder, air generated by a breathing of a user;
capturing, using a magnetometer and based on data captured using the magnetic encoder, a first portion of breath data;
capturing, using a microphone and based on data captured using the sound encoder, a second portion of breath data;
generating a first signal representation of the first portion of the breath data;
generating a second signal representation of the second portion of the breath data;
calculating, based on the first signal representation and the second signal representation, a flow rate for the breathing of the user; and
calculating, based on the flow rate and a cross sectional area of a breathing tube of the spirometric encoding device, a volume for the breathing of the user.

10. The computer-implemented method of claim 9, further comprising providing breathing instructions directing the user to breathe through the spirometric encoding device.

11. The computer-implemented method of claim 10, wherein the breathing instructions comprise multiple breathing sessions as part of a single breathing test.

12. The computer-implemented method of claim 9, wherein the first signal representation is generated based on calculating a Fourier transformation of the first portion of the breath data.

13. The computer-implemented method of claim 9, wherein the second signal representation is generated based on calculating a Fourier transformation of the second portion of the breath data.

14. The computer-implemented method of claim 9, wherein:
the second signal representation indicates a direction of air flow generated based on the second portion of the breath data; and
the second portion of the breath data comprises a first tone when the air flow is in a first direction and a second tone when the air flow is in a second direction.

15. A non-transitory computer readable medium storing instructions that, when executed by one or more processors, cause the one or more processors to perform steps comprising:
obtaining, using a spirometric encoding device comprising a spirometric encoding assembly comprising a magnetic encoder and a sound encoder, air generated by a breathing of a user;
capturing, using a magnetometer and based on data captured using the magnetic encoder, a first portion of breath data;
capturing, using a microphone and based on data captured using the sound encoder, a second portion of breath data;
generating a first signal representation of the first portion of the breath data;
generating a second signal representation of the second portion of the breath data;
calculating, based on the first signal representation and the second signal representation, a flow rate for the breathing of the user; and
calculating, based on the flow rate and a cross sectional area of a breathing tube of the spirometric encoding device, a volume for the breathing of the user.

16. The non-transitory computer readable medium of claim 15, wherein the instructions further cause the one or more processors to perform steps comprising providing breathing instructions directing the user to breathe through the spirometric encoding device.

17. The non-transitory computer readable medium of claim 16, wherein the breathing instructions comprise multiple breathing sessions as part of a single breathing test.

18. The non-transitory computer readable medium of claim 15, wherein the first signal representation is generated based on calculating a Fourier transformation of the first portion of the breath data.

19. The non-transitory computer readable medium of claim 15, wherein the second signal representation is generated based on calculating a Fourier transformation of the second portion of the breath data.

20. The non-transitory computer readable medium of claim 15, wherein:
the second signal representation indicates a direction of air flow generated based on the second portion of the breath data; and
the second portion of the breath data comprises a first tone when the air flow is in a first direction and a second tone when the air flow is in a second direction.
